(19) United States
(12) Patent Application Publication
(10) Pub. No.: US 2025/0122161 A1
(43) Pub. Date: Apr. 17, 2025

(54) PROCESS FOR PREPARING A OXACYLOHEXANE OR OXACYLOPENTANE DERIVATIVE

(71) Applicant: Firmenich SA, Satigny (CH)

(72) Inventors: Pierre-Yves DAPSENS, Satigny (CH); Lea VILCOQ, Satigny (CH)

(21) Appl. No.: 18/293,766

(22) PCT Filed: Jul. 28, 2022
(86) PCT No.: PCT/EP2022/071261

§ 371 Date: Jan. 30, 2024

(30) Foreign Application Priority Data
Jul. 30, 2021 (EP) 21188879.7

(51) Int. Cl. C07D 307/77; B01J 29/08; B01J 29/18; B01J 29/70

(52) U.S. Cl. CPC C07D 307/77; B01J 29/084; B01J 29/18; B01J 29/7007

(57) ABSTRACT

The present invention relates to the field of organic synthesis and, more specifically, it concerns a process for the preparation of an oxacylopentane or oxacylohexane derivative of formula (II) comprising the cyclodehydration of a 1,4- or 1,5-diol of formula (I) performed in the presence of a heterogenous acidic catalyst.

### TECHNICAL FIELD

The present invention relates to the field of organic synthesis and, more specifically, it concerns a process for the preparation of a oxacylopentane or oxacylohexane derivative of formula (II) comprising the cyclodehydration of a 1,4- or 1,5-diol of formula (I) performed in the presence of a heterogenous acidic catalyst.

### BACKGROUND

The oxacylopentane or oxacylohexane derivatives represent skeletons highly desirables which could be used as such or as key intermediates useful to prepare more complex compounds in different fields such as, among others, perfumery, cosmetic, pharmaceutic or agrochemistry. Relevant oxacyclopentane derivatives in perfumery industry are, for example, Cetalox® (3a,6,6,9a-tetramethyldodecahydronaphtho[2,1-b]furan; origin: Firmenich SA, Geneva, Switzerland) or Ambrox® ((3aR,5aS,9aS,9bR)-3a,6,6,9a-tetramethyldodecahydronaphtho[2,1-b]furan; origin: Firmenich SA, Geneva, Switzerland) being a key constituent of natural ambergris. Those perfuming ingredients represent some of the most sought-after ingredients in the perfumery industry. Several alternative processes to prepare Cetalox® or Ambrox® have been developed and in particular via a cyclodehydratation reaction of 1-(2-hydroxyethyl)-2,5,5,8a-tetramethyldecahydronaphthalen-2-ol as a last step.

In the meantime, today there is a need to foster sustainable processes, for examples using recyclable catalysts such as heterogenous catalysts. The cyclodehydratation reaction in a presence of heterogeneous catalysts, in particular to produce Cetalox® or Ambrox® from the corresponding diol, have already been reported mainly using clay as catalyst, for example, in U.S. Pat. No. 5,670,670 or in WO2013007832. However, said reaction has been exemplified only with a large amount of clay. In addition, clay is a natural material possessing properties depending on deposit and origin which could not be easily replicated and is a finite resource, e.g. some clay catalysts reported in U.S. Pat. No. 5,670,670 or in WO2013007832 are not anymore commercially available. The only example of cyclodehydratation reaction in a presence of synthetic heterogeneous catalysts with predictable and stable properties; i.e. different than clay, has been disclosed in US20100248316 wherein a large amount of basic zeolite is required.

So, there is still a need to develop a sustainable cyclodehydratation process in a presence of a small amount of heterogeneous reliable catalyst while maintaining a high conversion and selectivity.

The present invention allows to solve the above problem by using a heterogenous acidic catalyst in order to prepare a oxacylopentane or oxacylohexane derivative To the best of our knowledge, the invention's conditions have never been reported in the prior art.

### SUMMARY OF THE INVENTION

The invention relates to a novel process allowing the preparation of a oxacylopentane or oxacylohexane derivative by the cyclodehydration of a 1,4- or 1,5-diol in a presence of a small amount of a heterogeneous acidic catalyst never reported or suggested in the prior art.

So, a first object of the present invention is a process for the preparation of a oxacylopentane or oxacylohexane derivative of formula

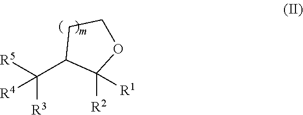

in the form of any one of its stereoisomers or a mixture thereof;
wherein m is 1 or 2;
wherein each R¹ and R² represent, when taken separately, independently from each other, a C₁₋₁₈ alkyl group optionally comprising one or two functional groups selected amongst ether, ester, carbonyl, amine, amide or alcohol group;
each R³ and R⁴ represent, when taken separately, independently from each other, a hydrogen atom or a C₁₋₁₈ alkyl group optionally comprising one or two functional groups selected amongst ether, ester, carbonyl, amine, amide or alcohol group; or R¹ and R², when taken together, represent a C₄₋₁₁ alkanediyl group optionally comprising one or two functional groups selected amongst ether, ester, carbonyl, amine, amide or alcohol group and/or R² and R³, when taken together, represent a C₂₋₁₈ alkanediyl group and/or R³ and R⁴, when taken together, represent a C₄₋₉ alkanediyl group and/or R¹ and R⁴, when taken together, represent a C₂₋₉ alkanediyl group; and
R⁵ represents a hydrogen atom or a C₁₋₃ alkyl group;
   comprising the cyclodehydration of a 1,4- or 1,5-diol of formula

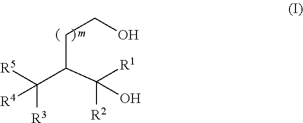

in the form of any one of its stereoisomers or a mixture thereof;
   wherein m, R¹, R², R³, R⁴ and R⁵ have the same meaning as defined for oxacylopentane or oxacylohexane derivative of formula (II);
   wherein the cyclodehydration is performed in the presence of an heterogenous acidic catalyst provided that
a) the heterogenous acidic catalyst is not a clay;
b) when the heterogeneous acidic catalyst is an aluminosilicate catalyst, the Silicon:Aluminum ratio is greater or equal to 3:1; and
c) the process is not carried out under supercritical conditions.

### DESCRIPTION OF THE INVENTION

Surprisingly, it has now been discovered that the cyclodehydration of a 1,4- or 1,5-diol may be performed in a presence of a small amount of a heterogenous acidic catalyst by selecting a different catalyst than clay while not compromising the selectivity and the conversion.

Therefore, a first object of the present invention is a process for the preparation of an oxacylopentane or oxacylohexane derivative of formula

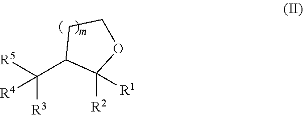

in the form of any one of its stereoisomers or a mixture thereof;
wherein m is 1 or 2;
wherein each R¹ and R² represent, when taken separately, independently from each other, a C₁₋₁₈ alkyl group optionally comprising one or two functional groups selected amongst ether, ester, carbonyl, amine, amide or alcohol group;
each R³ and R⁴ represent, when taken separately, independently from each other, a hydrogen atom or a C₁₋₁₈ alkyl group optionally comprising one or two functional groups selected amongst ether, ester, carbonyl, amine, amide or alcohol group; or R¹ and R², when taken together, represent a C₄₋₁₁ alkanediyl group optionally comprising one or two functional groups selected amongst ether, ester, carbonyl, amine, amide or alcohol group and/or R² and R³, when taken together, represent a C₂₋₁₈ alkanediyl group and/or R³ and R⁴, when taken together, represent a C₄₋₉ alkanediyl group and/or R¹ and R⁴, when taken together, represent a C₂₋₉ alkanediyl group; and
R⁵ represents a hydrogen atom or a C₁₋₃ alkyl group;
   comprising the cyclodehydration of a 1,4- or 1,5-diol of formula

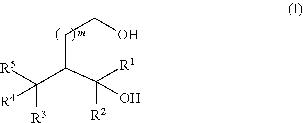

in the form of any one of its stereoisomers or a mixture thereof;
   wherein m, R¹, R², R³, R⁴ and R⁵ have the same meaning as defined for oxacylopentane or oxacylohexane derivative of formula (II);
   wherein the cyclodehydration is performed in the presence of an heterogenous acidic catalyst provided that
a) the heterogenous acidic catalyst is not a clay;
b) when the heterogeneous acidic catalyst is an aluminosilicate catalyst, the Silicon:Aluminum ratio is greater or equal to 3:1; and
c) the process is not carried out under supercritical conditions.

For the sake of clarity, by the term “heterogenous acidic catalyst” it is meant that the heterogeneous catalyst is not basic such as catalyst CBV100 or CBV320. In other words, the heterogenous acidic catalyst is not CBV100 or CBV320.

For the sake of clarity, by the term “oxacylopentane or oxacylohexane”, or the similar, it is meant the normal meaning understood by a person skilled in the art, i.e. oxacylopentane derivative is a tetrahydrofuran derivative and oxacylohexane derivative is a tetrahydropyran derivative.

For the sake of clarity, by the term “1,4- or 1,5-diol”, or the similar, it is meant the normal meaning understood by a person skilled in the art, i.e. a compound having at least two alcohol functional groups being separated by 4 or 5 carbon atoms; e.g. (OH)—CH₂—CH₂—CH₂—CH₂—(OH) or (OH)CH₂—CH₂—CH₂—CH₂—CH₂—(OH). The person skilled in the art is well aware that the cyclodehydration of 1,4-diol according to the invention's process provides an oxacylopentane derivative and the cyclodehydration of 1,5-diol according to the invention's process provides an oxacylohexane derivative.

For the sake of clarity, by the expression “any one of its stereoisomers or a mixture thereof”, or the similar, it is meant the normal meaning understood by a person skilled in the art, i.e. that the compounds cited in the invention can be a pure enantiomer or a mixture of enantiomers. In other words, the compounds cited in the invention may possess at least one stereocenter which can have two different stereochemistries (e.g. R or S), e.g. the R¹ group may comprise at least one stereocenter. Said compounds may even be in the form of a pure enantiomer or in the form of a mixture of enantiomers. The compounds cited in the invention may even be in the form of a pure diastereoisomer or in the form of a mixture of diastereoisomers when said compounds possess more than one stereocenter. Said compounds can be in a racemic form or scalemic form. Therefore, said compounds can be one stereoisomer or in the form of a composition of matter comprising, or consisting of, various stereoisomers.

The term “optionally” is understood that a group can or cannot be substituted by or comprise a certain functional group.

It is understood that with the terms “ . . . optionally comprising one or two functional groups selected amongst ether, ester, carbonyl, amine, amide or alcohol group . . . ” it is meant that the groups can either substitute a hydrogen atom of the alkyl group and thus be laterally attached to said alkyl group, or substitute a carbon atom (if chemically possible) of the alkyl group and thus be inserted into the alkyl chain. For example, a —CH₂—CH₂—CHOH—CH₂— group represents a C₄ alkyl group comprising an alcohol group (substitution of a hydrogen atom), a —CH₂—CH₂—COO—CH₂—CH₂—OCO—CH₂—CH₂— group represents a C₆ alkyl group comprising two ester groups (substitution of carbon atoms/insertion into the alkyl chain) and, similarly, a —CH₂—CH₂—O—CH₂—CH₂—O—CH₂—CH₂— group represents a C₆ alkyl group comprising two ether groups.

It is understood that by “ . . . R¹ and R², when taken together, represent a C₄₋₁₁ linear, branched or cyclic alkanediyl group . . . and/or R² and R³, when taken together, represent a C₂₋₁₈ alkanediyl group and/or R³ and R⁴, when taken together . . . and/or R¹ and R⁴, when taken together, represent” or the similar, that said group could form a (poly)cyclic alkyl group. In other words compound (I) could be acyclic, monocyclic, bicyclic or tricyclic, e.g. in the case wherein R² and R³, as well as R³ and R⁴, are taken together, the compound of formula (I) comprises a bicyclic moiety such as a decalin, e.g. R², R³ and R⁴, taken together, represents an alkanetriyl.

The terms “alkyl” and “alkanediyl” are understood as comprising linear, branched, cyclic or alicyclic alkyl and alkanediyl groups.

According to any embodiment of the present invention, the heterogenous acidic catalyst is not a clay.

According to any embodiment of the present invention, the heterogenous acidic catalyst may be amorphous or crystalline, particularly crystalline. The heterogenous acidic catalyst comprises silicon and a second metal selected from the group consisting of aluminum, boron, iron, tin, titanium, zirconium, hafnium and a mixture thereof. Particularly, the heterogenous acidic catalyst is an aluminosilicate catalyst. Particularly, the aluminosilicate catalyst is a Zeolite.

According to any embodiment of the present invention, the heterogenous acidic catalyst is free of alkaline or alkaline earth metals or lanthanides. Even more particularly, the heterogenous acidic catalyst is free of calcium, sodium potassium and/or lanthanum. The terms “free” is understood as the catalyst comprises less than 1 ppm, even less than 0.5 ppm, even less than 100 ppb, even less than 10 ppb, even less 1 ppb, even more the catalyst does not comprise alkaline or alkaline earth metals or lanthanide.

According to a particular embodiment of the present invention, the heterogenous acidic catalyst has a hydrophilic surface.

According to any embodiment of the present invention, the zeolite is a large pore zeolite.

By “the term large pore zeolite” it is meant the normal meaning in the art; i.e. 12 membered ring zeolite having pore size comprised in the range between 6.0 Angstrom and 8 Angstrom, particularly in the range between 6.0 Angstrom and 7.5 Angstrom.

According to any embodiment of the present invention, the zeolite has a FAU, BEA or MOR topology. Particularly, the zeolite has a FAU topology. Particularly, the zeolite do not have a MFI topology.

According to any embodiment of the present invention, the heterogeneous acidic catalyst is a dealuminated zeolite. Dealumination is conventionally understood as removal of aluminum atoms from a zeolite structure. Dealumination leads to an increase of the Silicon:Aluminum ratio of a zeolite. Non-limiting examples for suitable methods for dealumination known in the art are hydrothermal treatment, acidic treatment, treatment with gaseous halides or halogens or complexation with chelating agents or a combination of the as-mentioned treatments. A person skilled in the art is aware of these methods and of ways to perform them.

According to any embodiment of the present invention, the zeolite with a FAU topology is a dealuminated ultrastable Y-type (USY) zeolite. USY zeolites are typically prepared from Y-type zeolites to increase their stability and improve their catalytic activity by removing intra-framework aluminum with a combination of treatments comprising ion-exchange, steaming, acid leaching and calcination. Such treatments are for example described in the patents U.S. Pat. Nos. 5,601,798A and 4,477,336A and are well-known to a person skilled in the art.

According to any embodiment of the present invention, the Silicon:Aluminum ratio is greater or equal to 2.5:1, even greater or equal to 3:1, greater or equal to 5:1, even more greater or equal to 8:1.

According to any embodiment of the present invention, the Silicon:Aluminum ratio is comprised in the range between 2.5:1 and 300:1. Particularly, the Silicon:Aluminum ratio is comprised in the range between 3:1 and 300:1. Particularly, the Silicon:Aluminum ratio is comprised in the range between 5:1 and 300:1. Particularly, the Silicon:Aluminum ratio is comprised in the range between 5:1 and 150:1. Particularly, the Silicon:Aluminum ratio is comprised in the range between 10:1 and 150:1. Even more particularly, the Silicon:Aluminum ratio is comprised in the range between 10:1 and 70:1.

According to any embodiment of the present invention, the zeolite is used in its protonic form. The latter can be directly provided by the manufacturer and used as such or obtained by calcination of the ammonium-exchange form if required. Ideally, any sample should be preactivated before reaction. The preactivation may be carried out by heating the zeolite at a temperature comprised between 300° C. and 600° C. for at least 1 hour.

The heterogenous acidic catalyst can be added into the reaction medium of the invention's process to form a oxacylopentane or oxacylohexane derivative of formula (II) in a large range of concentrations. As non-limiting examples, one can cite, as heterogenous acidic catalyst concentration values those ranging from 0.5 wt % to 20 wt %, relative to the total amount of the 1,4- or 1,5-diol. Particularly, the heterogenous acidic catalyst concentration may be comprised between 1 wt % to 15 wt %. Even more particularly, the heterogenous acidic catalyst concentration may be comprised between 3 wt % to 10 wt %. It goes without saying that the process works also with more catalyst. However the optimum concentration of heterogenous acidic catalyst will depend, as the person skilled in the art knows, on the nature of the latter, on the nature of the substrate, on the temperature and on the desired time of reaction.

The heterogenous acidic catalyst is commercially available compound or can be prepared by several methods, such as the one reported in US20040141911, U.S. Pat. Nos. 6,054,113, and 4,840,930.

Non-limiting examples of suitable heterogenous acidic catalyst may include CBV720 (Origin Zeolyst), CBV760 (Origin Zeolyst) CBV780 (Origin Zeolyst), HSZ-385HUA (Origin Zeolyst), CBV21A (Origin Zeolyst), HSZ-640HOA (Origin Tosoh), CP814E* (Origin Zeolyst), CP814C* (Origin Zeolyst) or CP811C-300 (Origin Zeolyst).

According to any one of the invention's embodiments, the invention's process for the preparation of a oxacylopentane or oxacylohexane derivative of formula (II) is not carried out under supercritical conditions; i.e. using supercritical fluid, such as for example supercritical water or supercritical CO₂.

According to any one of the invention's embodiments, the invention's process for the preparation of a oxacylopentane or oxacylohexane derivative of formula (II) is carried out at a temperature comprised between 0° C. and 150° C. In particular, the temperature is in the range between 30° C. and 70° C. Of course, a person skilled in the art is also able to select the preferred temperature as a function of the melting and boiling point of the starting and final products as well as the desired time of reaction, conversion or selectivity.

The invention's process for the preparation of a oxacylopentane or oxacylohexane derivative of formula (II) can be carried out in the presence or absence of a solvent. When a solvent is required or used for practical reasons, then any solvent current in such reaction type can be used for the purposes of the invention. Non-limiting examples include C₆₋₁₂ aromatic solvents such as xylene, toluene, 1,3-diisopropylbenzene, cumene pseudocumene, anisole or chlorobenzene or mixtures thereof, hydrocarbon solvents such as cyclohexane, heptane or mixtures thereof, nitrile solvent such as acetonitrile, esteral solvents such as ethyl acetate or ethereal solvents such as tetrahydrofuran, diethyether, methyl tetrahydrofuran or mixtures thereof. The choice of the solvent is function of the nature of the substrate and/or catalyst and the person skilled in the art is well able to select the solvent most suitable in each case to optimize the reaction.

The invention's process for the preparation of a oxacylopentane or oxacylohexane derivative of formula (II) is carried out under batch or continuous conditions.

Water is formed during the invention's process. Said water may be removed from the reaction mixture, for example by azeotropic distillation or by performing the invention's process under a slight vacuum.

The invention's process for the preparation of a oxacylopentane or oxacylohexane derivative of formula (II) may be performed under atmospheric pressure or under a slight vacuum.

According to any one of the invention's embodiments, the 1,4- or 1,5-diol comprise a primary alcohol group and a tertiary alcohol group.

According to any one of the above embodiments of the invention, said the 1,4- or 1,5-diol of formula (I) is a C₉-C₂₀ compound.

According to any one of the invention's embodiments, m may be 1. In other word, compound of formula (I) is a 1,4-diol and compound of formula (II) is a oxacyclopentane derivative.

According to any one of the invention's embodiments, R² and R³ may be taken together, and represent a C₃₋₁₈ alkanediyl group. Particularly, R² and R³ may be taken together, and represent a C₃₋₁₈ linear or branched alkanediyl group.

According to any one of the invention's embodiments, the 1,4-diol is a compound of formula

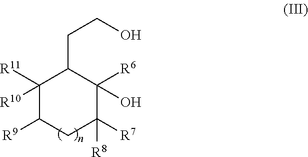

in the form of any one of its stereoisomers or a mixture thereof;
wherein n is 0 or 1;
each R⁷, R⁸, R⁹, R¹⁰ represent, when taken separately, independently from each other, a hydrogen atom or a C₁₋₉ alkyl group optionally comprising one or two functional groups selected amongst ether, ester, carbonyl, amine, amide or alcohol group; R⁶ represents a C₁₋₉ alkyl group optionally comprising one or two functional groups selected amongst ether, ester, carbonyl, amine, amide or alcohol group; or
R⁶ and R⁷, when taken together, represent a C₃₋₉ linear or branched alkanediyl group optionally comprising one or two functional groups selected amongst ether, ester, carbonyl, amine, amide or alcohol group and/or R⁸ and R⁹, when taken together, represent a C₁₋₂ linear alkanediyl group and/or R⁹ and R¹⁰, when taken together, represent a C₃₋₁₀ linear or branched alkanediyl group and/or R⁷ and R¹⁰, when taken together, represent a C₁₋₃ linear or branched alkanediyl group; and
R¹¹ represents a hydrogen atom or a C₁₋₃ linear or branched alkyl group.

According to any one of the invention's embodiments, the oxacylopentane derivative is a compound of formula

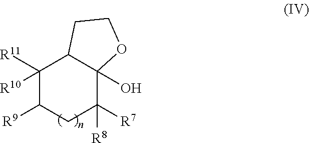

in the form of any one of its stereoisomers or a mixture thereof;
wherein n is 0 or 1;
each R⁷, R⁸, R⁹, R¹⁰ represent, when taken separately, independently from each other, a hydrogen atom or a C₁₋₉ alkyl group optionally comprising one or two functional groups selected amongst ether, ester, carbonyl, amine, amide or alcohol group; R⁶ represents a C₁₋₉ alkyl group optionally comprising one or two functional groups selected amongst ether, ester, carbonyl, amine, amide or alcohol group; or
R⁶ and R⁷, when taken together, represent a C₃₋₉ linear or branched alkanediyl group optionally comprising one or two functional groups selected amongst ether, ester, carbonyl, amine, amide or alcohol group and/or R⁸ and R⁹, when taken together, represent a C₁₋₂ linear alkanediyl group and/or R⁹ and R¹⁰, when taken together, represent a C₃₋₁₀ linear or branched alkanediyl group and/or R⁷ and R¹⁰, when taken together, represent a C₁₋₃ linear or branched alkanediyl group; and
R¹¹ represents a hydrogen atom or C₁₋₃ linear or branched alkyl group.

According to a particular embodiment compounds of formula (III) may be monocyclic, bicyclic or tricyclic compounds and compounds of formula (IV) may be bicyclic or tricyclic compounds. Preferably, compounds of formula (III) may be monocyclic or bicyclic compounds and compounds of formula (IV) may be bicyclic or tricyclic compounds. Even more preferably, compounds of formula (III) may be bicyclic compounds and compounds of formula (IV) may be tricyclic compounds. Said compound of formula (III) can be synthetic or natural.

According to any one of the invention's embodiments, R⁷, R⁸, R⁹, R¹⁰ may represent, when taken separately, independently from each other, a hydrogen atom or a C₁₋₉ alkyl group optionally comprising one or two functional groups selected amongst ether, ester, carbonyl, amine, amide or alcohol group; R⁶ may represent a C₁₋₉ alkyl group optionally comprising one or two functional groups selected amongst ether, ester, carbonyl, amine, amide or alcohol group; or R⁶ and R⁷, when taken together, may represent a C₃₋₉ linear or branched alkanediyl group optionally comprising one or two functional groups selected amongst ether, ester, carbonyl, amine, amide or alcohol group and/or R⁸ and R⁹, when taken together, may represent a C₁₋₂ linear alkanediyl group and/or R⁹ and R¹⁰, when taken together, may represent a C₃₋₉ linear or branched alkanediyl group and/or R⁷ and R¹⁰, when taken together, may represent a C₁₋₃ linear or branched alkanediyl group. Particularly, R⁷, R⁸, R⁹, R¹⁰ may represent, when taken separately, independently from each other, a hydrogen atom or a C₁₋₆ alkyl group optionally comprising one or two functional groups selected amongst ether, ester, carbonyl, amine, amide or alcohol group; R⁶ may represent a C₁₋₉ alkyl group optionally comprising one or two functional groups selected amongst ether, ester, carbonyl, amine, amide or alcohol group; or R⁶ and R⁷, when taken together, may represent a C₃₋₈ linear or branched alkanediyl group optionally comprising one or two functional groups selected amongst ether, ester, carbonyl, amine, amide or alcohol group and/or R⁸ and R⁹, when taken together, may represent a C₁₋₂ linear alkanediyl group and/or R⁹ and R¹⁰, when taken together, may represent a C₃₋₈ linear or branched alkanediyl group and/or R⁷ and R¹⁰, when taken together, may represent a C₁₋₃ linear or branched alkanediyl group. Particularly, R⁷, R⁸, R⁹, R¹⁰ may represent, when taken separately, independently from each other, a hydrogen atom or a C₁₋₄ alkyl group optionally comprising one or two functional groups selected amongst ether, ester, carbonyl, amine, amide or alcohol group; R⁶ may represent a C₁₋₉ alkyl group optionally comprising one or two functional groups selected amongst ether, ester, carbonyl, amine, amide or alcohol group; or R⁶ and R⁷, when taken together, may represent a C₃₋₈ linear or branched alkanediyl group optionally comprising one or two functional groups selected amongst ether, ester, carbonyl, amine, amide or alcohol group and/or R⁸ and R⁹, when taken together, may represent a C₁₋₂ linear alkanediyl group and/or R⁹ and R¹⁰, when taken together, may represent a C₃₋₈ linear or branched alkanediyl group and/or R⁷ and R¹⁰, when taken together, may represent a C₁₋₃ linear or branched alkanediyl group.

According to any one of the invention's embodiments, said R⁷ group may represent a hydrogen atom or C₁₋₃ alkyl group. Particularly, R⁷ group may represent a hydrogen atom or a methyl group. Even more particularly, R⁷ group may represent a hydrogen atom.

According to any one of the invention's embodiments, said R⁸ group may represent a hydrogen atom or C₁₋₃ alkyl group. Particularly, R⁸ group may represent a hydrogen atom or a methyl group. Even more particularly, R⁸ group may represent a hydrogen atom.

According to any one of the invention's embodiments, said R⁹ group may represent a hydrogen atom or C₁₋₃ alkyl group. Particularly, R⁹ group may represent a hydrogen atom or a methyl group. Even more particularly, R⁹ group may represent a hydrogen atom.

According to any one of the invention's embodiments, said R¹⁰ group may represent a hydrogen atom or C₁₋₃ alkyl group. Particularly, R¹⁰ group may represent a hydrogen atom or a methyl group. Even more particularly, R¹⁰ group may represent a hydrogen atom.

According to any one of the invention's embodiments, said R⁶ and R⁷ when taken together, may represent a C₃₋₆ linear or branched alkanediyl group or even preferably a C₄ branched alkanediyl group.

According to any one of the invention's embodiments, said R⁸ and R⁹ when taken together, may represent a C₁₋₂ linear alkanediyl group or even preferably a C₂ linear alkanediyl group.

According to any one of the invention's embodiments, said R⁹ and R¹⁰, when taken together, may represent a C₃₋₆ linear or branched alkanediyl group or even preferably a C₆ branched alkanediyl group.

According to any one of the invention's embodiments, said R⁷ and R¹⁰, when taken together, may represent a C₃ branched alkanediyl group.

According to any one of the invention's embodiments, n may be 1.

According to any one of the invention's embodiments, the 1,4-diol may be a compound of formula

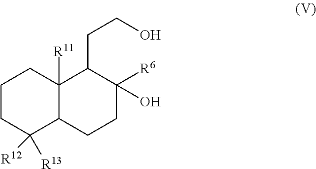

in the form of any one of its stereoisomers or a mixture thereof;
wherein each R⁶ and R¹¹ have the same meaning as defined above and R¹² and R¹³ represent, independently from each other, a C₁₋₃ linear or branched alkyl group.

According to any one of the invention's embodiments, the oxacylopentane derivative is a compound of formula

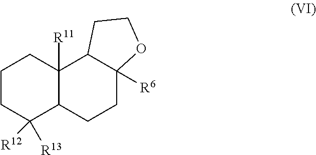

in the form of any one of its stereoisomers or a mixture thereof;
wherein each R⁶, R¹¹, R¹² and R¹³ have the same meaning as defined above.

According to any one of the invention's embodiments, R⁶ may be a C₁₋₆ alkyl group optionally comprising one or two functional groups selected amongst ether, ester, carbonyl, amine, amide or alcohol group. Particularly, R⁶ may be a C₁₋₄ alkyl group optionally comprising one or two functional groups selected amongst ether, ester and carbonyl group. Particularly, R⁶ may be a C₁₋₃ linear, or branched alkyl group optionally comprising one or two functional groups selected amongst ether, ester and carbonyl group. Particularly, R⁶ may be a methyl, ethyl or n-propyl group. Even more particularly, R⁶ may be a methyl group.

According to any one of the invention's embodiments, R¹¹ may be a C₁₋₃ linear alkyl group. Particularly, R¹¹ may be a methyl or ethyl group. Even more particularly, R¹¹ may be a methyl group.

According to any one of the invention's embodiments, R¹² may be methyl or ethyl group. Even more particularly, R¹² may be a methyl group.

According to any one of the invention's embodiments, R¹³ may be methyl or ethyl group. Even more particularly, R¹³ may be a methyl group.

According to a particular embodiment of the invention, the 1,4-diol may be 1-(2-hydroxyethyl)-2,5,5,8a-tetramethyldecahydronaphthalen-2-ol and the corresponding the oxacylopentane derivative may be 3a,6,6,9a-tetramethyldodecahydronaphtho[2,1-b]furan which have four stereogenic centers being in a configuration R or S or a mixture thereof. In other worlds 1-(2-hydroxyethyl)-2,5,5,8a-tetramethyldecahydronaphthalen-2-ol may be in the form of an essentially pure stereoisomer or in the form of a mixture of stereoisomers. According to a particular embodiment of the invention, compound (I), (III) or (V) may be 1-(2-hydroxyethyl)-2,5,5,8a-tetramethyldecahydronaphthalen-2-ol in the form of a mixture of stereoisomers containing at least 80% of both stereoisomers (1RS,2RS,4aSR,8aSR)-1-(2-hydroxyethyl)-2,5,5,8a-tetramethyldecahydronaphthalen-2-ol and (1RS,2SR,4aSR,8aSR)-1-(2-hydroxyethyl)-2,5,5,8a-tetramethyldecahydronaphthalen-2-ol. Particularly, compound (I), (III) or (V) may be 1-(2-hydroxyethyl)-2,5,5,8a-tetramethyldecahydronaphthalen-2-ol in the form of a mixture of stereoisomers containing at least 50% of (1RS,2RS,4aSR,8aSR)-1-(2-hydroxyethyl)-2,5,5,8a-tetramethyldecahydronaphthalen-2-ol. Even more particularly, compound (I), (III) or (V) may be 1-(2-hydroxyethyl)-2,5,5,8a-tetramethyldecahydronaphthalen-2-ol in the form of a mixture of stereoisomers containing at least 75% of (1RS,2RS,4aSR,8aSR)-1-(2-hydroxyethyl)-2,5,5,8a-tetramethyldecahydronaphthalen-2-ol. According to a particular embodiment of the invention, compound (I), (III) or (V) may be (1RS,2RS,4aSR,8aSR)-1-(2-hydroxyethyl)-2,5,5,8a-tetramethyldecahydronaphthalen-2-ol. For the sake of clarity, by the expression “1RS,2RS,4aSR,8aSR” it is meant an equimolar mixture of 1R,2R,4aS,8aS and 1S,2S,4aR,8aR and by the expression “1RS,2SR,4aSR,8aSR” it is meant an equimolar mixture of 1R,2S,4aS,8aS and 1S,2R,4aR,8aSR. According to particular embodiment of the invention, compound (I), (III) or (V) may be (1R,2R,4aS,8aS)-1-(2-hydroxyethyl)-2,5,5,8a-tetramethyldecahydronaphthalen-2-ol.

According to any embodiment of the invention's, the invention's process is stereoselective. In other words, the cyclodehydration of 1,4-diol (1R,2R,4aS,8aS)-1-(2-hydroxyethyl)-2,5,5,8a-tetramethyldecahydronaphthalen-2-ol provides (3aR,5aS,9aS,9bR)-3a,6,6,9a-tetramethyldodecahydronaphtho[2,1-b]furan.

The compound of formula (I), (III) (V) can be prepared by several methods known in the art, for 1-(2-hydroxyethyl)-2,5,5,8a-tetramethyldecahydronaphthalen-2-ol, said compound may be obtained by the hydrogenation of sclareolide as reported in WO2019175158.

Typical manners to execute the invention's process are reported herein below in the examples.

### EXAMPLES

The invention will now be described in further detail by way of the following examples, wherein the abbreviations have the usual meaning in the art, the temperatures are indicated in degrees centigrade (° C.). The preparation of. Gas chromatography was performed on an Agilent 7890 A Series equipped with a HP5 column (30 m×0.25 mm ID, 0.25 μm film) and tetradecane was used as internal standard.

### Example 1

### Typical Experimental Procedure for the Preactivation of the Zeolite Catalyst:

### a) Under Static Air

10 g of the as-received zeolite as the one reported in Table I was placed in a muffle furnace (static air) and heated at 10° C. min⁻¹ to 550° C. for 3 h.

### b) Under Flowing Gas

30 kg of as-received zeolite as the one reported in Table I, in particular CBV780, were placed in a tubular oven and heated at 5° C. min⁻¹ to 550° C. for 3 h under flowing nitrogen or air (50 mL/min).

### Example 2

General Cyclodehydration Procedure to Prepare a Oxacylopentane Starting From of 1,4-diol

(1S,2R,4aS,8aS)-1-(hydroxymethyl)-2,5,5,8a-tetramethyldecahydronaphthalen-2-ol (1,4-diol, 80 g, 0.317 mol) was charged in a 500 ml reactor, diluted with toluene (320 g, 3.47 mol) at 20 wt. % and then heated to 50° C. Once the desired temperature was reached, a preactivated zeolite (6.42 g—preactivation performed as reported in example 1) from table I was added and the reaction stirred for 6 h affording up to 93.5% of the desired (3aR,5aS,9aS,9bR)-3a,6,6,9a-tetramethyldodecahydronaphtho[2,1-b]furan product (oxacylopentane). The results of the dehydrocyclization upon using various zeolites topologies and structures are reported in Table I evidencing the key role of zeolite acidity and topology.

TABLE I
Preparation of a oxacylopentane by cyclodehydration
of 1,4-diol in a presence of different

| | | | | Time | oxacylopentane | 1,4-diol |
|---|---|---|---|---|---|---|
| Name | Topology | Ion | Si/A1 | h | GC % | GC % |
| CBV100^{1) and 2)} | FAU | Na⁺ | 2.55:1 | 6 | 7.0 | 91.7 |
| CBV400¹⁾ | FAU | H⁺ | 2.55:1 | 2 | 13.9 | 84.5 |
| CBV320^{1) and 3)} | FAU | Ca⁺⁺ | 2.55:1 | 2 | 82.6 | 0.0 |
| CBV320^{1) and 4)} | FAU | Ca⁺⁺ | 2.55:1 | 6 | 17.5 | 78.3 |
| CBV720 | FAU | H⁺ | 15:1 | 6 | 68.8 | 23.0 |
| CBV760 | FAU | H⁺ | 30:1 | 4 | 68.9 | 24.9 |
| | | | | 6 | 91.9 | 0.3 |
| CBV780 | FAU | H⁺ | 40:1 | 4 | 86.4 | 5.9 |
| | | | | 6 | 93.0 | 0.0 |
| CBV21A | MOR | NH₄⁺⁵⁾ | 10:1 | 4 | 85.9 | 4.6 |
| | | | | 6 | 91.9 | 0.47 |
| HSZ-640HOA | MOR | H⁺ | 9:1 | 6 | 86.66 | 5.29 |
| CP814E* | BEA | NH4⁺⁵⁾ | 12.5:1 | 4 | 92.5 | 0.0 |
| | | | | 6 | 93.4 | 0.0 |
| CP814C* | BEA | NH₄⁺⁵⁾ | 19.5:1 | 4 | 68.6 | 23.1 |
| | | | | 6 | 85.7 | 4.1 |
| CP811C-300 | BEA | H⁺ | 150:1 | 6 | 65.9 | 25.7 |

¹⁾Comparative examples
²⁾Starting NaY used to prepare CBV320 as described in US20100248316
³⁾as described in US20100248316: room temperature with 3 wt. % of 1,4-Diol in hexane and using 900 wt. % of catalyst
⁴⁾The prior art basic heterogeneous catalyst does not allow obtaining high conversion with 8wt % of catalyst and with 20 wt. % of 1,4-Diol contrary to the invention's process
⁵⁾H⁺ after preactivation

### Example 3

Cyclodehydration to Prepare a Oxacylopentane Starting From of 1,4-diol Using Different Solvents

Example 2 was repeated using preactivated CBV780 and performing the reaction in various solvents as encompass in Table II.

TABLE II
Cyclodehydration to prepare a oxacylopentane starting
from of 1,4-diol using different solvents

| | oxacylopentane | 1,4-diol |
|---|---|---|
| Solvent | GC % | GC % |
| Dichloromethane³⁾ | 87.1 | 0 |
| Toluene | 93.5 | 0 |
| Anisole | 92.7 | 0 |
| Chlorobenzene | 95.5 | 0 |
| Pseudocumene | 92.5 | 0 |
| Dimethylcarbonate | 80.1 | 0 |
| Dioxane | 73.2 | 0 |
| Tetrahydrofurane | 61.2 | 0 |

6) Reaction performed at 25° C.

### Example 4

Cyclodehydration to Prepare a Oxacylopentane Starting From of 1,4-diol Using Under Reduced Pressure

Example 2 was repeated using 4 g of CBV780 (5 wt. %) and performing the reaction under reduced pressure for 12 h to remove progressively the water in-situ formed. After filtration and separation by distillation, an isolated yield of 90.0 mol. % of (3aR,5aS,9aS,9bR)-3a,6,6,9a-tetramethyldodecahydronaphtho[2,1-b]furan at 99.9 GC % was attained.

### Example 5

Cyclodehydration to Prepare a Oxacylopentane Starting From of 1,4-diol Using Not-Preactivated CBV780

Example 2 was repeated using 5 wt. % of not-preactivated CBV780 (as-received) and performing the reaction at 70° C. for 4 h. Under those conditions, 88% of the desired (3aR,5aS,9aS,9bR)-3a,6,6,9a-tetramethyldodecahydronaphtho[2,1-b]furan was obtained. The reaction can be performed as well at 50° C. for 24 h yielding 90 GC % of the desired oxacyclopentane

### Example 6

Cyclodehydration to Prepare a Oxacylopentane Starting From of 1,4-diol Using Strongly Acidic Ion Exchange Resin in Their Dry State.

(1S,2R,4aS,8aS)-1-(hydroxymethyl)-2,5,5,8a-tetramethyldecahydronaphthalen-2-ol (1,4-diol, 80 g, 0.317 mol) was charged in a 500 ml reactor, diluted with toluene (320 g, 3.47 mol) at 20 wt. % and then heated to 50° C. Once the desired temperature was reached, Amberlyst A-15 dry (4 g) was added and the reaction stirred for 6 h affording up to 69.7% of the desired (3aR,5aS,9aS,9bR)-3a,6,6,9a-tetramethyldodecahydronaphtho[2,1-b]furan product (oxacylopentane). Similar results are obtained upon the use of Amberlyst A-35 dry.

1. A process for the preparation of an oxacylopentane or oxacylohexane derivative of formula 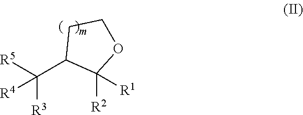
in the form of any one of its stereoisomers or a mixture thereof;
wherein m is 1 or 2;
wherein each R¹ and R² represent, when taken separately, independently from each other, a C₁₋₁₈ alkyl group optionally comprising one or two functional groups selected amongst ether, ester, carbonyl, amine, amide or alcohol group; each R³ and R⁴ represent, when taken separately, independently from each other, a hydrogen atom or a C₁₋₁₈ alkyl group optionally comprising one or two functional groups selected amongst ether, ester, carbonyl, amine, amide or alcohol group; or R¹ and R², when taken together, represent a C₄₋₁₁ alkanediyl group optionally comprising one or two functional groups selected amongst ether, ester, carbonyl, amine, amide or alcohol group and/or R² and R³, when taken together, represent a C₂₋₁₈ alkanediyl group and/or R³ and R⁴, when taken together, represent a C₄₋₉ alkanediyl group and/or R¹ and R⁴, when taken together, represent a C₂₋₉ alkanediyl group; and
R⁵ represents a hydrogen atom or a C₁₋₃ alkyl group;
comprising the cyclodehydration of a 1,4- or 1,5-diol of formula 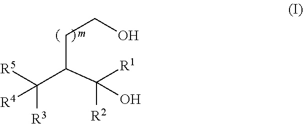
in the form of any one of its stereoisomers or a mixture thereof;
wherein m, R¹, R², R³, R⁴ and R⁵ have the same meaning as defined for oxacylopentane or oxacylohexane derivative of formula (II);
wherein the cyclodehydration is performed in the presence of an heterogenous acidic catalyst provided that
a) the heterogenous acidic catalyst is not a clay;
b) when the heterogeneous acidic catalyst is an aluminosilicate catalyst, the Silicon:Aluminum ratio is greater or equal to 3:1; and
c) the process is not carried out under supercritical conditions.

2. The process according to claim 1, wherein the heterogenous acidic catalyst is crystalline.

3. The process according to claim 1, wherein the heterogenous catalyst comprises silicon and a second metal selected from the group consisting of aluminum, boron, iron, tin, titanium, zirconium, hafnium and a mixture thereof.

4. The process according to claim 1, wherein the heterogenous acidic catalyst is free of calcium, potassium, sodium and/or lanthanum.

5. The process according to claim 1, wherein the heterogenous acidic catalyst is an aluminosilicate catalyst.

6. The process according to claim 1, wherein the aluminosilicate catalyst is a zeolite.

7. The process according to claim 1, wherein the zeolite is a large pore zeolite.

8. The process according to claim 1, wherein the zeolite has a FAU, BEA or MOR topology.

9. The process according to claim 1, wherein the Silicon:Aluminum ratio is comprised in the range between 3:1 and 300:1.

10. The process according to claim 1, wherein the zeolite with a FAU topology is a dealuminated ultrastable Y-type (USY) zeolite.

11. The process according to claim 1, wherein the zeolite is used in its protonic form.

12. The process according to claim 1, wherein m is 1.

13. The process according to claim 1, wherein the 1,4-diol is a compound of formula 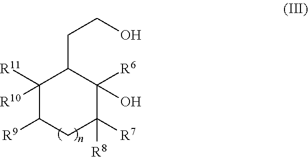
in the form of any one of its stereoisomers or a mixture thereof;
wherein n is 0 or 1;
each R⁷, R⁸, R⁹, R¹⁰ represent, when taken separately, independently from each other, a hydrogen atom or a C₁₋₉ alkyl group optionally comprising one or two functional groups selected amongst ether, ester, carbonyl, amine, amide or alcohol group; R⁶ represents a C₁₋₉ alkyl group optionally comprising one or two functional groups selected amongst ether, ester, carbonyl, amine, amide or alcohol group; or
R⁶ and R⁷, when taken together, represent a C₃₋₉ linear or branched alkanediyl group optionally comprising one or two functional groups selected amongst ether, ester, carbonyl, amine, amide or alcohol group and/or R⁸ and R⁹, when taken together, represent a C₁₋₂ linear alkanediyl group and/or R⁹ and R¹⁰, when taken together, represent a C₃₋₁₀ linear or branched alkanediyl group and/or R⁷ and R¹⁰, when taken together, represent a C₁₋₃ linear or branched alkanediyl group; and
R¹¹ represents a hydrogen atom or a C₁₋₃ linear or branched alkyl group.

14. The process according to claim 1, wherein the oxacylopentane derivative is a compound of formula 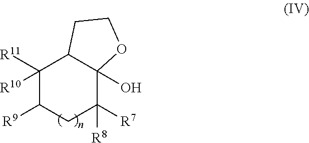
in the form of any one of its stereoisomers or a mixture thereof;
wherein n is 0 or 1;
each R⁷, R⁸, R⁹, R¹⁰ represent, when taken separately, independently from each other, a hydrogen atom or a C₁₋₉ alkyl group optionally comprising one or two functional groups selected amongst ether, ester, carbonyl, amine, amide or alcohol group; R⁶ represents a C₁₋₉ alkyl group optionally comprising one or two functional groups selected amongst ether, ester, carbonyl, amine, amide or alcohol group; or
R⁶ and R⁷, when taken together, represent a C₃₋₉ linear or branched alkanediyl group optionally comprising one or two functional groups selected amongst ether, ester, carbonyl, amine, amide or alcohol group and/or R⁸ and R⁹, when taken together, represent a C₁₋₂ linear alkanediyl group and/or R⁹ and R¹⁰, when taken together, represent a C₃₋₁₀ linear or branched alkanediyl group and/or R⁷ and R¹⁰, when taken together, represent a C₁₋₃ linear or branched alkanediyl group; and
R¹¹ represents a hydrogen atom or a C₁₋₃ linear or branched alkyl group.

15. The process according to claim 1, wherein the 1,4-diol is a compound of formula 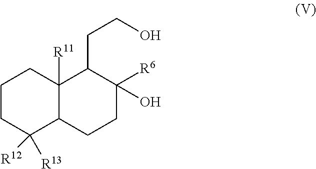
in the form of any one of its stereoisomers or a mixture thereof;
wherein R⁶ represents a C₁₋₉ alkyl group optionally comprising one or two functional groups selected amongst ether, ester, carbonyl, amine, amide or alcohol group;
R¹¹ represents a hydrogen atom or a C₁₋₃ linear or branched alkyl group;
and each R¹² and R¹³ represent, independently from each other, a C₁₋₃ linear or branched alkyl group.

16. The process according to claim 9, wherein the Silicon:Aluminum ratio is comprised in the range between 5:1 and 300:1.
